(19) United States
(12) Patent Application Publication
(10) Pub. No.: US 2017/0065561 A1
(43) Pub. Date: Mar. 9, 2017

(54) METHODS OF CANCER THERAPY

(71) Applicant: CAMBRIDGE ENTERPRISE LIMITED, Cambridge, Cambridgeshire (GB)

(72) Inventors: Timothy Cox, Cambridge (GB); Elena Pavlova, Cambridge (GB)

(73) Assignee: CAMBRIDGE ENTERPRISE LIMITED, Cambridge, Cambridgeshire (GB)

(21) Appl. No.: 15/308,467

(22) PCT Filed: May 1, 2015
(86) PCT No.: PCT/GB2015/051302

§ 371 Date: Nov. 2, 2016

(30) Foreign Application Priority Data
May 2, 2014 (GB) 1407837.2

(51) Int. Cl. A61K 31/4025

(52) U.S. Cl. CPC A61K 31/4025

(57) ABSTRACT

This invention relates to the treatment of lymphoproliferative cancers, such as myeloma and lymphoma, using UDP-glucosylceramide synthase inhibitors, such as eliglustat tartrate. Methods, uses and inhibitors for use are provided.

### FIELD OF INVENTION

This invention relates to the treatment of lymphoproliferative cancers, such as myeloma and lymphoma.

### BACKGROUND OF INVENTION

Multiple myeloma is the most frequent primary malignancy involving the skeleton and has an age-adjusted incidence of 4.5-6.9 per 100,000 women and men, respectively [1]. While several environmental factors, including ionizing radiation-exposure are associated with this disorder, few genetic risk factors for myeloma are recognized, apart from an increased incidence in persons of African ancestry.

However, multiple myeloma and B cell lymphoma are the most frequent causes of cancer-related death in individuals with the inborn error of glycosphingolipid metabolism, Gaucher disease. A high frequency of multiple myeloma has been noted in numerous studies and may be increased up to 50-fold [2-9]. Persistent monoclonal gammopathy of undetermined significance and hypergammaglobulinaemia often occurs in adults with Gaucher disease; the paraproteins also persist despite enzyme therapy that ameliorates the principal haematopoietic manifestations (10, 11, 12). It has been assumed that life-long accumulation of glycosphingolipid in the alternatively-activated macrophages that characterize this disorder [10, 13-16], leads to the release of cytokines and related molecules which stimulate clonal expansion of B lymphocytes and plasma cells—ultimately inducing the appearance of cognate malignancies [2, 10, 11, 13, 17, 18, 19]. However, the molecular signals that link disturbed lysosomal recycling of cellular glycosphingolipids in Gaucher disease with B cell proliferation and carcinogenesis [20, 21], are unknown.

There is a marked increase in the age-related incidence of spontaneous B cell lymphoma/myeloma in an authentic mouse model of Gaucher disease. Of particular note, this phenomenon is accompanied by the appearance of monoclonal paraproteins—thus faithfully mimicking the comorbid evolution of Gaucher disease in human individuals (22). Greatly increased β-glucosylsphingosine concentrations are found in the circulation of this inducible disease model with β-glucosidase-deficiency.

In Gaucher disease, deficiency of the lysosomal acid hydrolase, glucosylceramidase (E.C.3.2.1.45), resulting from mutations in the human GBA1 gene, causes the accumulation of β-D-glucosylceramides and their nonacylated analogue, β-D-glucosylsphingosine. These glycosphingolipids arise from the digestion of complex globosides and gangliosides present in cell membranes. The first committed step in the biosynthesis of neutral glycosphingolipids is catalysed by UDP-glucose: N-acylsphingosine transferase (UDP-glucosylceramide synthase) which leads to formation of β-D-glucosylceramides. This enzyme is the pharmacological target for the stratagem of substrate deprivation therapy: by partially inhibiting de novo synthesis of glucosylceramides, the imbalance between their formation and breakdown is corrected and the pathological storage of undegraded macromolecules that characterizes Gaucher disease, can be ameliorated. In individuals with chronic forms of Gaucher disease with residual glucosylceramidase activity due to co-inheritance of non-inactivating GBA 1 alleles, as proposed by Radin, judicious pharmacological inhibition of glucosylceramide biosynthesis allows the imbalance between the formation of this essential precursor molecule and its degradation to be corrected [23, 24]. This precept provides the basis for the use of inhibitors of UDP-glucosyltransferase as an alternative treatment for Gaucher disease.

The first clinical inhibitor of UDP-glucosylceramide transferase, approved for the treatment of type 1 (non-neuronopathic) Gaucher disease was the iminosugar, N-butyldeoxynojirimycin (miglustat) [25, 26]. However, several clinical studies indicate a small therapeutic action and unwanted effects that may impede general acceptance of this agent alternative to enzyme therapy [27, 28].

Lately, eliglustat tartrate (GENZ-112638), a ceramide analogue with a potent and selective inhibitory action on UDP-glucosylceramide transferase, has been developed for non-neuronopathic Gaucher disease, and licensed as eliglustat tartrate. The compound is structurally similar to D-threo-1-phenyl-2-decanoylamino-3-morpholino-propanol (PDMP) [29, 30].

The action of eliglustat tartrate (GENZ-112638) in a model of murine Gaucher disease has been analyzed in a (GBA^{D409/null}) knock-in model of Gaucher disease which shows a mild clinical phenotype: 10 week-old and 7 month-old mice received the compound at either 75 or 150 mg/kg daily orally for 10 weeks [31, 32]. A dose-dependent reduction of glucosylceramide accompanied by decreased infiltration of abnormal macrophages in tissues was reported.

Eliglustat tartrate appears so far to be safe and tolerable in healthy adults [33]; in Phase III clinical trials over two years, oral administration of eliglustat tartrate to individuals with non-neuronopathic Gaucher disease clearly improved key clinical manifestations of the disease with few unwanted effects [34, 35].

### SUMMARY OF THE INVENTION

The present inventors have investigated the effect of glucosylceramide biosynthesis inhibitors on lymphoproliferative and other tissue responses using non-neuronopathic Gaucher disease mice that undergo conditional deletion of the endogenous GBA1 gene function in haematopoietic tissues [22] as a model. Inhibition of UDP-glucosylceramide synthase was found to suppress clonal B cell proliferation and to reduce the occurrence of myeloma and B-cell lymphoma. UDP-glucosylceramide synthase inhibitors may therefore be useful in the suppression of abnormal or malignant lymphoproliferation and the treatment of lymphoproliferative cancers, such as myeloma and lymphoma.

An aspect of the invention provides a method of suppressing abnormal or malignant lymphoproliferation or treating a lymphoproliferative cancer comprising; administering a UDP-glucosylceramide synthase inhibitor to an individual in need thereof.

Another aspect of the invention provides a UDP-glucosylceramide synthase inhibitor for use in the suppression of abnormal or malignant lymphoproliferation or treatment of lymphoproliferative cancer in an individual.

Another aspect of the invention provides the use of a UDP-glucosylceramide synthase inhibitor in the manufacture of a medicament for use in the suppression of abnormal or malignant lymphoproliferation or the treatment of lymphoproliferative cancer in an individual.

UDP-glucosylceramide synthase inhibitors that may be used in accordance with the invention include eliglustat tartrate.

### BRIEF DESCRIPTION OF FIGURES

FIG. 1 shows the eliglustat tartrate (GENZ-112638) study protocol. Upper panel shows the main treatment experiment—early oral administration of Genz-112638 at a dose 300 mg/kg/day in 22 GD mice during defined periods; lower panel shows the delayed treatment experiment—delayed administration of Genz-112638 at a dose 300 mg/kg/day in 8 GD mice from age 6 months for 6 months.

FIG. 2 shows plasma glucosylceramide (GL-1) levels in Gaucher mice treated with eliglustat tartrate (GENZ-112638). The drug is clearly shown to decrease the concentration of glucosylceramide in these short-term experiments.

FIG. 3 shows the plasma concentrations of glucosylsphingosine in Gaucher mice treated with eliglustat tartrate (GENZ-112638). The deacylated form of glucosylceramide (glucosylsphingosine) in plasma of GD mice treated with Genz-112638 was reduced in ˜5-fold (p<0.01) after 6 months and in ˜2-fold after 9 months of treatment (p<0.05); Median of plasma glucosylsphingosine in GD mice with the intermittent treatment was not significantly different when compared with age matched untreated GD mice.

FIG. 4 shows the plasma concentrations of glucosylceramide in Gaucher mice treated with eliglustat tartrate (GENZ-112638). Glucosylceramide concentration in plasma of GD mice after 10 months treatment was reduced (p<0.01); increased plasma concentration in the group of GD mice with intermittent treatment of Genz-112638 for 10 months.

FIG. 5 shows spleen and liver sections of GD mice treated and untreated with Genz-112638 and age matched untreated control stained with mouse macrophage marker Mac-3: (A, D)—the control spleen and liver sections. (B, E) the spleen and liver sections of an untreated 10-months old GD mouse showed strong Mac-3 positive staining demonstrating Mac-3 immunoreactive macrophages. Insertions in B and E are higher magnifications of Mac-3 positive enlarged multinucleated macrophages usually joined in units. (C, F) the spleen and liver sections of a 13-months old GD mouse received Genz-112638 for 10 months.

FIG. 6 shows spleen sections of untreated, Genz-112638 treated GD mice and age-matched control mice stained with pan B cell (CD45R/B220), pan T cell (CD3) and plasma cell (CD138) markers: (i, iv) untreated control spleen—white pulp shows normal localization of B and T cell cells; (ii, v) untreated GD mouse with white pulp lymphoid hyperplasia and invasion of germinal centre by centrocytes positively stained with anti-mouse B220 antibodies; (iii, vi) Genz-112638 treated GD mouse spleen sections—note the preserved white pulp architecture with distinct B and T cells localization; (vii) control spleen section stained with plasma cell marker (CD138); (viii) increased number of CD138⁺ lymphocytes (plasma cells) usually found in red pulp of untreated GD mice; (ix) decreased number of CD138⁺ lymphocytes in spleen of GD mice treated with Genz-112638 for 10 months.

FIG. 7A shows the effect of Genz-112638 on secreted monoclonal paraproteins. Serum/plasma immunofixation protein electrophoresis in untreated and Genz-112638 treated GD mice at late time points: (i) plasma sample of a 12 month-old untreated GD mouse with IgA A monoclonal paraprotein; (ii) serum sample obtained from 14 month-old untreated GD mouse with IgG K monoclonal immunoglobulin; (iii) serum sample of untreated 19 month-old GD mouse with IgM K monoclonal paraprotein. The early administration of Genz-112638 prevents development of monoclonal paraproteins in GD mice: (iv, v) plasma samples of GD mice after 9-10 months treatment with GENZ-112638 showing normal diffused staining pattern in each of the five anti-immunoglobulin sera lanes; (vi) control age-matched plasma sample.

FIG. 7B shows microscopic sections of spleen of GD mice stained with the proliferation marker Ki-67 in the region of the white pulp: (i) untreated GD mouse showing lymphoid hyperplasia and increased Ki-67 staining; (ii) spleen section of GD mouse treated with Genz-112638 for 10 months with decreased population of lymphoid cells.

FIG. 7C shows total plasma IgG concentrations in untreated GD mice and GD mice treated with GENZ-112638.

### DETAILED DESCRIPTION OF THE INVENTION

The experiments described herein show that inhibition of UDP-glucosylceramide synthase reduces or inhibits lymphoproliferation, in particular abnormal or malignant lymphoproliferation, in individuals and elicits a beneficial anti-cancer effect. The invention, in various aspects, relates to the suppression of lymphoproliferation and the treatment of lymphoproliferative cancer using UDP-glucosylceramide synthase inhibitors.

Lymphoproliferative cancers are characterized by the abnormal or malignant proliferation of lymphocytes in an individual, often resulting in the formation of a tumour or tissue mass.

Lymphoproliferative cancers may be characterised by abnormal or malignant proliferation of B lymphocytes, T lymphocytes, plasma cells, or precursors thereof. Lymphoproliferative cancers may include lymphomas, such as B-cell lymphoma, and multiple myeloma—a malignant proliferation of plasma cells, which belong to the lineage of B-cell lymphocytes.

In some embodiments, cancers suitable for treatment as described herein may be characterised by paraproteinemia or hyperglobulinaemia, such as monoclonal gammopathy. Paraproteinemia or hyperglobulinaemia may be reduced by treatment as described herein.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or leporid) may be employed.

In some embodiments, the individual may have minimal residual disease (MRD) after an initial cancer treatment.

An individual with lymphoproliferative cancer may display at least one identifiable sign, symptom, or laboratory finding that is sufficient to make a diagnosis of a lymphoproliferative cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a lymphoproliferative cancer in an individual may include identification of a particular cell type (e.g. a cancer cell) in a sample of a body fluid or tissue obtained from the individual. In some embodiments, the individual may have been previously identified or diagnosed with lymphoproliferative cancer or a method of the invention may comprise identifying or diagnosing lymphoproliferative cancer in the individual for example by determining the presence of an identifiable sign, symptom, or laboratory finding indicative of cancer in the individual.

In some embodiments, an individual suitable for treatment may not display abnormal lipid metabolism or have a lipid storage disorder, such as Gaucher disease, or have been diagnosed with such a disorder. The lymphoproliferative cancer may be a non-Gaucher related lymphoproliferative cancer.

In other embodiments, an individual suitable for treatment may have a lipid storage disorder, such as Gaucher disease, or have been diagnosed with such a disorder or disease. For example, the lymphoproliferative cancer may be a Gaucher related lymphoproliferative cancer.

UDP-glucosylceramide synthase (EC 2.4.1.80: UDP-glucose: N acylsphingosine D-glucosyltransferase; GlcCer synthase) is involved in the biosynthesis of neutral glycosphingolipids and catalyses the formation of β-D-glucosylceramides. Inhibition of UDP-glucosylceramide synthase reduces glucosylsphingosine levels in the individual. For example, the glucosylceramide:ceramide ratio in the individual may be reduced by UDP-glucosylceramide synthase inhibition. In some preferred embodiments, excess glucosylsphingosine synthesis may be reduced to non-excessive physiological levels, such that ceramide levels may be unaffected by the inhibition of UDP-glucosylceramide synthase.

Without being bound by theory, it is believed that glucosylsphingosine stimulates downstream signaling pathways leading to the systemic malignant proliferation of lymphocytes and lymphocyte precursors, such as B cells and B cell precursors. Reduction of glucosylsphingosine concentrations and production through the inhibition of UDP-glucosylceramide synthase reduces the stimulation of these signaling pathways and thereby reduces or prevents malignant proliferation. For example, the proliferative drive for B cells may abrogated by UDP-glucosylceramide synthase inhibition so that monoclonal expansion and malignancy are reduced or prevented.

A UDP-glucosylceramide synthase inhibitor is an exogenous factor that inhibits the activity of UDP-glucosylceramide synthase. Suitable UDP-glucosylceramide synthase inhibitors include small organic chemical compounds or molecules.

Compounds that inhibit UDP-glucosylceramide synthase may be identified using standard in vitro or ex vivo assays. For example, cell homogenates may be incubated with UDP-³H Glucose and liposomes comprising octanoylsphingosine, dioleoylphosphatidylcholine and/or sodium sulphatide in the presence and absence of the compound and the production of labelled UDP-glucosylceramide determined (see for example, refs [29] and [54]).

In other embodiments, expression of UDP-glucosylceramide synthase may be analysed at the mRNA or at protein level and compounds that reduce expression identified. Suitable techniques are well-known in the art.

Suitable UDP-glucosylceramide synthase inhibitors may have a specific selective inhibitory action towards UDP-glucosylceramide synthase in vitro and display minimal inhibitory properties towards glycosidases and other sphingolipid metabolism enzymes. In some embodiments, a UDP-glucosylceramide synthase inhibitor for use as described herein may inhibit UDP-glucosylceramide synthase with an IC₅₀ of less than 100 nM, preferably less than 50 nM.

Suitable UDP-glucosylceramide synthase inhibitors include iminosugars, such as N-butyldeoxynojirimycin (miglustat; Zavesca™, Actelion Pharmaceuticals), N-butyl deoxygalactonojirimyicin and more preferably D-threo-1-phenyl-2-decanoylamino-3-morpholino-propanol (PDMP) and analogues of PDMP.

Examples of suitable PDMP analogues^{29, 54} include: DL-threo-1-phenyl-2-palmitoylamino-3-pyrrolidino-1-propanol (P4); D-threo-1-(4′-hydroxy)phenyl-2-palmitoylamino-3-pyrrolidino-1-propanol (4′hydroxy-P4); and D-threo-1-(3,4-ethylenedioxy)phenyl-2-palmitoylamino-3-pyrrolidino-1-propanol (EtDO-P4).

Other suitable UDP-glucosylceramide synthase inhibitors are well-known in the art.

Preferred UDP-glucosylceramide synthase inhibitors inhibit the synthesis of β-D-glucosylceramide without affecting intracellular concentrations of ceramide, which may be toxic at high concentrations. For example, preferred UDP-glucosylceramide synthase inhibitors may inhibit UDP-glucosylceramide synthase without inhibiting 1-O-acylceramide synthase or other enzymes that metabolise ceramide⁶³. Inhibiting the de novo synthesis of glucosylceramide without increasing intracellular ceramide concentrations may be helpful, for example, in reducing or minimising pro-apoptotic effects in non-malignant cells.

In some preferred embodiments, the UDP-glucosylceramide synthase inhibitor is eliglustat tartrate (bis{N-[(1R,2R)-2-(2,3-dihydro-1,4-benzodioxin-6-yl)-2-hydroxy-1-(pyrrolidin-1-ylmethyl)ethyl]octanamide} (2R,3R)-2,3-dihydroxybutanedioate; GENZ-112638; Cerdelga™)^{29-34, 49} or an analogue thereof.

Eliglustat tartrate is selective for eliglustat tartrate and does not inhibit 1-O-acylceramide synthase or increase intracellular ceramide levels.

Preferred UDP-glucosylceramide synthase inhibitors may reduce or abolish UDP-glucosylceramide synthase activity in a cell to the same or greater extent than eliglustat tartrate under the same conditions. For example, preferred inhibitors may have a potency that is equal to or greater than the potency of eliglustat tartrate (i.e. an IC₅₀ of 24 nM or lower).

While it is possible for UDP-glucosylceramide synthase inhibitors, such as eliglustat tartrate, to be administered to the individual alone, it is preferable to present the compound in a pharmaceutical composition or formulation.

A pharmaceutical composition may comprise, in addition to the UDP-glucosylceramide synthase inhibitor, one or more pharmaceutically acceptable carriers, adjuvants, excipients, diluents, fillers, buffers, stabilisers, preservatives, lubricants, or other materials well-known to those skilled in the art. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well-known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The UDP-glucosylceramide synthase inhibitor or pharmaceutical compositions comprising the UDP-glucosylceramide synthase inhibitor may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the oral route, although other routes such as intraperitoneal, subcutaneous, transdermal, intravenous, nasal, intramuscular or other convenient routes are not excluded.

The pharmaceutical compositions comprising the active compounds may be formulated in a dosage unit formulation that is appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients.

Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Optionally, other therapeutic or prophylactic agents may be included in the pharmaceutical composition or formulation.

Inhibition of UDP-glucosylceramide synthase as described herein is useful in the treatment of cancer, for example lymphoproliferative cancers, such as B cell lymphoma and myeloma.

Treatment may be any treatment or therapy, whether of a human or an animal (e.g. in veterinary applications), in which some desired therapeutic effect is achieved, for example, the inhibition or delay of the onset or progress of the condition, and includes a reduction in the rate of progress, a halt in the rate of progress, inhibition of metastasis, amelioration of the condition, cure or remission (whether partial or total) of the condition, preventing, delaying, abating or arresting one or more symptoms and/or signs of the condition or prolonging survival of a subject or individual beyond that expected in the absence of treatment.

Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Inhibition of UDP-glucosylceramide synthase in an individual with cancer, as described herein may improve the capacity of the individual to resist cancer growth, in particular growth of a cancer already present the subject and/or decrease the propensity for cancer growth in the individual.

Treatment as described herein may include prophylactic treatment (i.e. prophylaxis) i.e. the individual being treated may not have or may not be diagnosed as having a lymphoproliferative cancer at the time of treatment. For example, an individual susceptible to or at risk of the occurrence or re-occurrence of lymphoproliferative cancer may be treated as described herein. Such treatment may prevent or delay the occurrence or re-occurrence of lymphoproliferative cancer in the individual or reduce its symptoms or severity after occurrence or re-occurrence. In some embodiments, the individual may have been previously identified as having increased susceptibility or risk of lymphoproliferative cancer compared to the general population or a method may comprise identifying an individual who has increased susceptibility or risk of lymphoproliferative cancer. Prophylactic or preventative treatment may be preferred in some embodiments.

UDP-glucosylceramide synthase inhibitors may be administered as described herein in therapeutically-effective amounts.

The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

The appropriate dosage of an active compound may vary from individual to individual. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the individual. The amount of active compounds and route of administration will ultimately be at the discretion of the physician, although generally the dosage will be to achieve therapeutic plasma concentrations of the active compound without causing substantial harmful or deleterious side-effects.

In general, a suitable dose of the active compound is in the range of about 100 μg to about 400 mg per kilogram body weight of the subject per day, preferably 200 μg to about 200 mg per kilogram body weight of the subject per day. Where the active compound is a salt, an ester, prodrug, or the like, the amount administered is calculated on the basis of the parent compound and so the actual weight to be used is increased proportionately. For example, 50 to 100 mg of eliglustat tartrate may be orally administered twice daily in capsule or tablet form.

A UDP-glucosylceramide synthase inhibitor, such as eliglustat tartrate, may be orally administered in an amount sufficient to maintain the serum concentration at a level that yields >50% inhibition of UDP-glucosylceramide synthase.

Administration in vivo can be effected in one dose, continuously or intermittently (e.g., in divided doses at appropriate intervals).

Methods of determining the most effective means and dosage of administration are well known in the art and will vary with the formulation used for therapy, the purpose of the therapy, the target cell being treated, and the subject being treated. Single or multiple administrations can be carried out with the dose level and pattern being selected by the physician.

Multiple doses of the UDP-glucosylceramide synthase inhibitor may be administered, for example 2, 3, 4, 5 or more than 5 doses may be administered. The administration of the UDP-glucosylceramide synthase inhibitor may continue for sustained periods of time. For example treatment with the UDP-glucosylceramide synthase inhibitor may be continued for at least 1 week, at least 2 weeks, at least 3 weeks, at least 1 month or at least 2 months. Treatment with the UDP-glucosylceramide synthase inhibitor may be continued for as long as is necessary to reduce cancer symptoms or achieve complete remission.

The UDP-glucosylceramide synthase inhibitor may be administered alone or in combination with other treatments, either simultaneously or sequentially dependent upon the individual circumstances. For example, a UDP-glucosylceramide synthase inhibitor as described herein may be administered in combination with one or more additional active compounds.

For example, a UDP-glucosylceramide synthase inhibitor as described herein may be administered in combination with an immunosuppressive agent.

Suitable immunosuppressive agents are well-known in the art and include glucocorticoids, such as cortisol and dexamethasone; alkylating agents, such as chlorambucil and cyclophosphamide; antimetabolites, such as methotrexate and azathioprine; antibodies, such as anti-TNF, anti-CD3 and anti-CD25 antibodies, for example infliximab and daclizumab; immunophilin binding compounds, such as cyclosporin, rapamycin and tacrolimus; and other immunosuppressants, such as fingolimod and myriocin.

Other aspects of the invention provide a method of immunosuppression comprising; administering a UDP-glucosylceramide synthase inhibitor as described above to an individual in need thereof and a UDP-glucosylceramide synthase inhibitor for use in immunosuppression in an individual.

A suitable individual may have or be at risk of aberrant, abnormal or malignant lymphoproliferation, as described herein.

Other aspects of the invention provide a method of treatment of hepatocellular carcinoma, renal adenocarcinoma or myeloid leukaemia comprising; administering a UDP-glucosylceramide synthase inhibitor as described above to an individual in need thereof and a UDP-glucosylceramide synthase inhibitor for use in hepatocellular carcinoma, renal adenocarcinoma or myeloid leukaemia in an individual.

The hepatocellular carcinoma, renal adenocarcinoma or myeloid leukaemia may be Gaucher related or non-Gaucher related.

Various further aspects and embodiments of the present invention will be apparent to those skilled in the art in view of the present disclosure.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### EXPERIMENTS

### 1. Methods

### Experimental Animals

The inducible mouse model of type 1 Gaucher disease [Gba^{tm1Karl/tm1Karl}Tg (Mx1-cre) 1Cgn/0 (MGI:3687965; MGI:2176073)[41]. The mating, induction with polyinosinic-polycytidylic (poly I:C) acid sodium salt solution and genotyping were conducted as previously described [22]. Animals of genotype Gba^{tm1Karl/tm1Karl}Tg (Mx1-cre) 1Cgn/0 are referred GD mice; induced mice with the Gba^{tm1Karl/tm1Karl} and Gba^{tm1Karl/+} genotypes and background strain wild type animals served as age-matched controls. All mice here reported had been bred on a C57BL/6 and 129/sv background.

Treatment with GENZ 112638

### Short-Term Dose-Range Tolerability Experiments

The drug GENZ 112638 (Genzyme) was formulated in a standard rodent food (TestDiet) at 0.075% and 0.15% w/w. A group of six GD mice received 150 mg/kg daily, a second group of six GD mice received the drug at 300 mg/kg daily, and eight age-sex matched GD animals were given a base diet (TestDiet). The animals received the drug for 14 days and were then killed for analysis of tissue lipids.

### Main Treatment Experiments

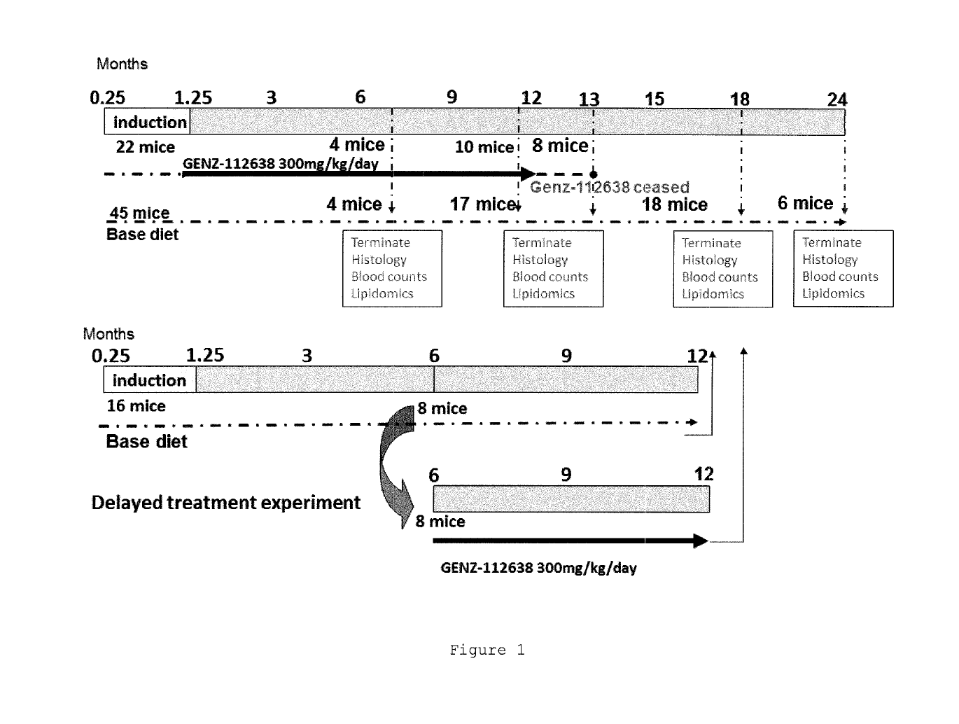

A total of 22 induced Gaucher disease mice received GENZ 112638 at 300 mg/kg daily: four mice received the treatment over 3 months, four mice received the drug for 6 months, six mice received the diet for 7-9 months. Animals were sacrificed at the end of the defined periods (FIG. 1). A group of 8 mice received the drug for 10 months and were killed at 14 months of age. To serve as a control group, 45 age- and sex-matched induced Gaucher mice were given a standard diet. All the animals were killed at the same intervals (FIG. 1).

GENZ 112638 was introduced at a mean age of 1.5 months, with access to the diet and water ad libitum. All experimental animals were housed in individually ventilated cages in groups of up to three mice within the same environmental conditions. The health of the animals was checked daily; body weights and food consumption were monitored at intervals of three days. The humane endpoints were approved and established: (i) reduction of 20% or more of the greatest recorded body weight or (ii) the development of adverse manifestations (e.g. development of frank tumours or cachexia).

### Delayed Treatment Experiments

To investigate the potential effects of delayed treatment, eight Gaucher mice were exposed to Genz-112638 after 6 months of age. The animals received the drug for 6 months at a dose 300 mg/kg daily and were killed at 12 months of age (FIG. 1). A group of eight matched Gaucher disease control mice were given the base standard diet; these animals were monitored and otherwise treated identically with those animals with delayed exposure to the study drug.

These regulated experimental studies were authorized by the UK Home Office under license and conducted exactly as approved under the Animals Scientific Procedures Act, (1986).

### Tissue and Blood Collection

Visceral organs (spleen, liver, lungs and kidneys) as well as long bones, lymph nodes, and, where relevant, tumours, were collected by dissection and either frozen or fixed in 4% w/v paraformaldehyde or 10% buffered formalin and processed in paraffin-embedded blocks for further analysis. Sample of long bones were fixed in 4% w/v paraformaldehyde and decalcified in EDTA for two weeks. Spleen and liver weights were recorded fresh at post mortem. Samples of venous blood were collected from the tail vein at 3 monthly intervals during the experimental protocol. Complete blood counts (CBC) were determined using an automated haematology analyser (SYSMEX XT 2000i). EDTA plasma or serum samples collected at post mortem were stored at −80° C. until thawed for analysis.

### Histology and Immunohistochemistry

Paraffin-embedded sections of 3.5 μm were stained routinely with haematoxylin and eosin. Immunohistochemical staining was carried out using rat anti-mouse CD45R/B220 (R&D systems), anti-mouse CD3 (Abcam), CD138 (BD, Pharmingen), rat anti-mouse Mac-3 M3/84 clone (BD, Pharmingen) and rabbit anti-mouse Ki-67 (Abcam) primary antibodies. Staining was realized by the avidin-biotin peroxidase technique (Vectastatin ABC elite kit), and developed using diaminobenzidine with cresyl violet or haematoxylin counterstaining.

Lymphoproliferation in serial sections of spleen, liver, lungs and kidneys was estimated by a scoring system modified as described by Elmore [42]. Sections were reviewed blindly for lymphoma and lymphoproliferative reactions by a board-certified veterinary pathologist.

### Plasma/Serum Protein Electrophoresis and Immunofixation

Plasma/serum protein electrophoresis was carried out in agarose gels using a semiautomatic system (Hydrasys, Sebia) using 10 μL samples of neat serum or plasma. Immunofixation using polyclonal anti-sera raised against murine heavy and light immunoglobulin chains was conducted with diluted serum samples as previously reported [22].

### Total IgG Plasma Concentrations

Total IgG ELISA kit (Affimetrix) was used to determine plasma IgG concentrations in selected groups of animals.

### Glycosphingolipid Concentrations

Concentrations of β-D-glucosylceramide in plasma in the short-term tolerability study were conducted as previously published [31, 32]. β-D-glucosylceramide, β-D-glucosylsphingosine and ceramide concentrations in tissues and plasma samples of the principal intervention and delayed-treatment experiments were quantified as previously reported [22].

### Statistics

The non-parametric Mann-Whitney test was used for between-groups comparisons. The Fisher's exact test was used for comparison of the occurrence of lymphoma or plasma paraprotein in treated and untreated groups. Statistical analyses were conducted using GraphPad Prism v5.0 software.

### 2. Results

### Dose-Ranging Studies of Genz-112638

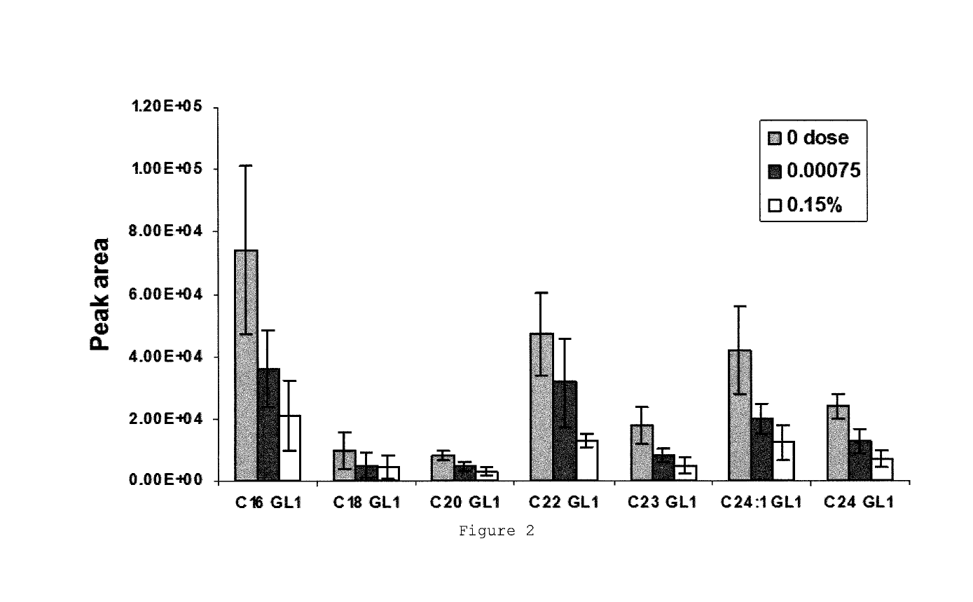

The groups of 6 week-old GD mice received 150 mg/kg and 300 mg/kg of GENZ 112638 orally for two weeks. A dose-dependent decrease of plasma glucosylceramide concentration was observed in these animals after two weeks of treatment (FIG. 2). The mice appeared to tolerate the drug well at both dosages without significant change in body weight over this period.

### Effect of Genz-112638 on Glycosphingolipid Accumulation

GD mice of approximately 1.5 months of age received GENZ 112638 at 300 mg/kg daily for 2 to 10 months. Generally these young animals tolerated the treatment. Two animals were withdrawn early from the study as a result of weight loss that was attributed to decreased food consumption. Although mice receiving GENZ 112638 had a lower average weight gain than animals without treatment over the prolonged period of this study, no unusual external features, behavioural changes or ill effects were apparent.

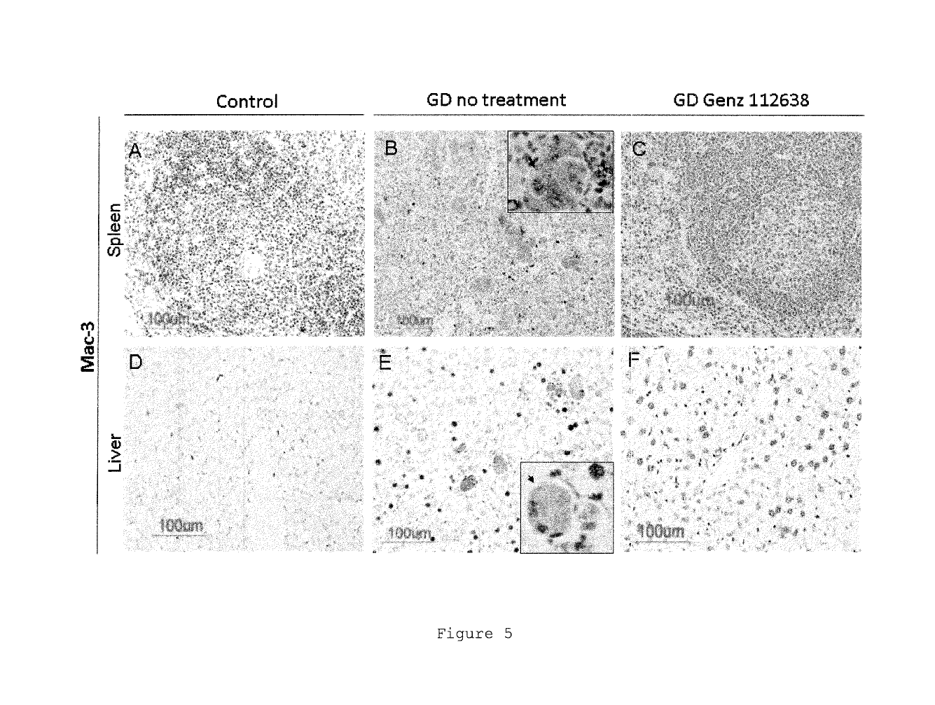

Gaucher cells were observed in the liver and spleen of the mice with conditional deletion of GBA1 in haematopoietic tissues, these appeared at approximately 10 months of age [22, 41). As predicted, histological examination of tissue sections obtained from animals receiving the interventional agent showed fewer lipid-engorged macrophages (FIG. 5 B, E). Furthermore, pathological macrophages staining for the Mac 3 antigen were markedly decreased or absent in tissues of GD mice that had received GENZ 112638 (FIGS. 5 C, F).

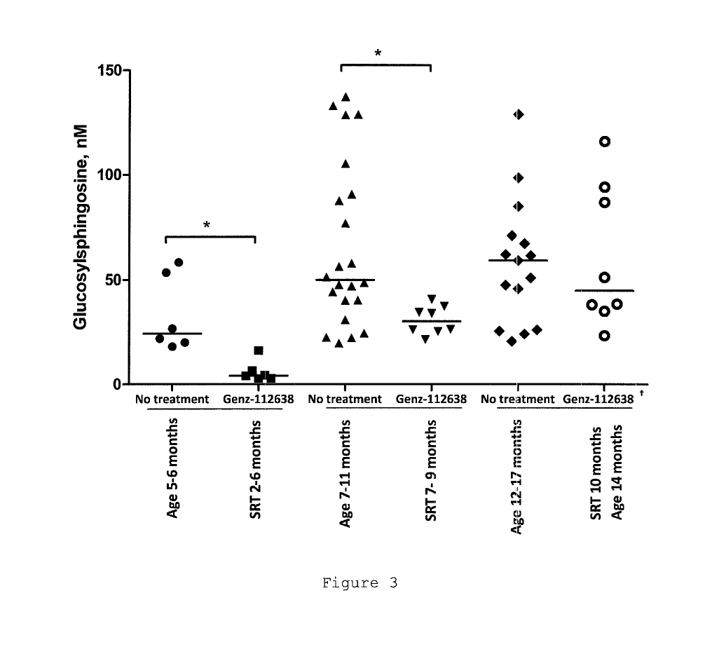

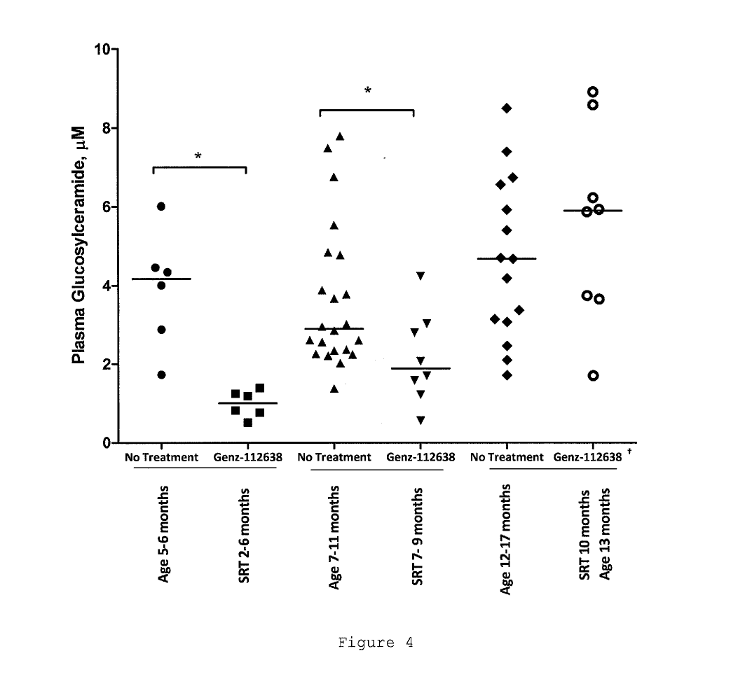

We have previously reported that concentrations of the unacylated lysoglycosphingolipid, β-D-glucosylsphingosine, were greatly elevated in plasma obtained from these Gaucher disease mice, while β-D-glucosylceramide concentrations were not raised. After treatment with GENZ 112638, plasma concentrations of these glycosphingolipids were decreased (FIG. 3, 4). It is noteworthy that in 8 GD mice in which exposure to the agent was intermittent, the mean β-D-glucosylceramide and β-D-glucosylsphingosine concentrations in plasma were raised above the reference range for healthy, age- and sex-matched non-Gaucher disease control animals; they were also greater than in the Gaucher mice that had received long term continuous treatment with GENZ 112638 (FIGS. 3,4). Mean plasma ceramide concentrations in animals receiving the high dose of GENZ 112638 were slightly increased. The glucosylceramide/ceramide ratio was markedly diminished in GD animals that had received GENZ 112638 compared with those animals that were not treated.

### Effects on B Cell Lymphoma and Monoclonal Paraprotein Secretion

No B cell lymphomas or other tumours were found in 22 Gaucher mice that had been exposed to GENZ 112638 for 2-10 months (table 3). In contrast, as previously described [22], in 14 out of 61 mice with identically induced Gaucher disease that had received the standard diet alone, frank B cell lymphoma developed.

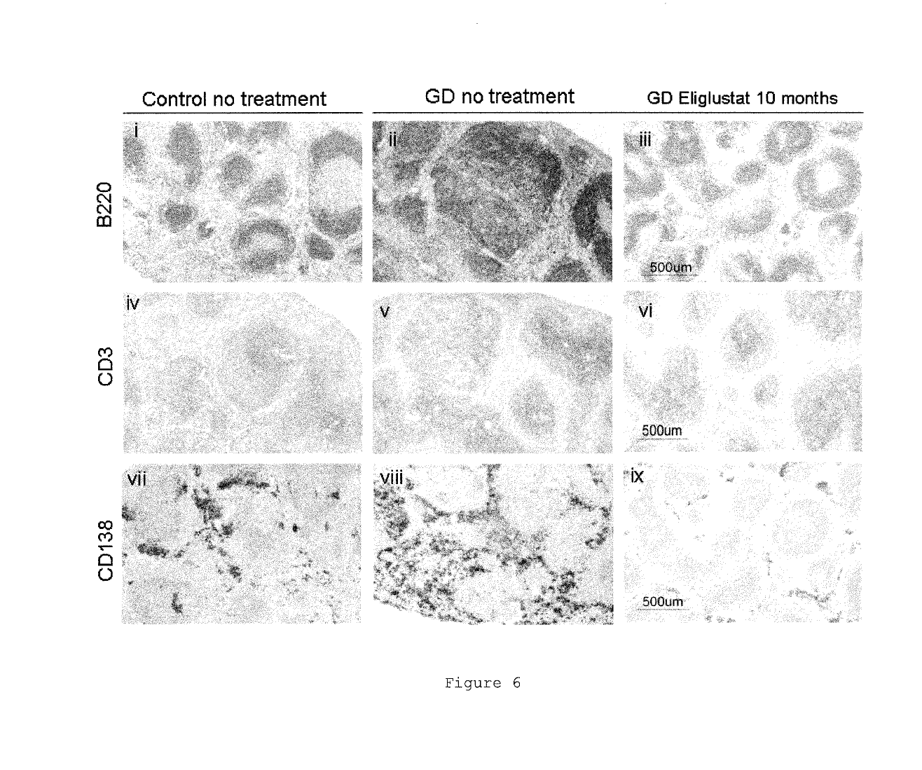

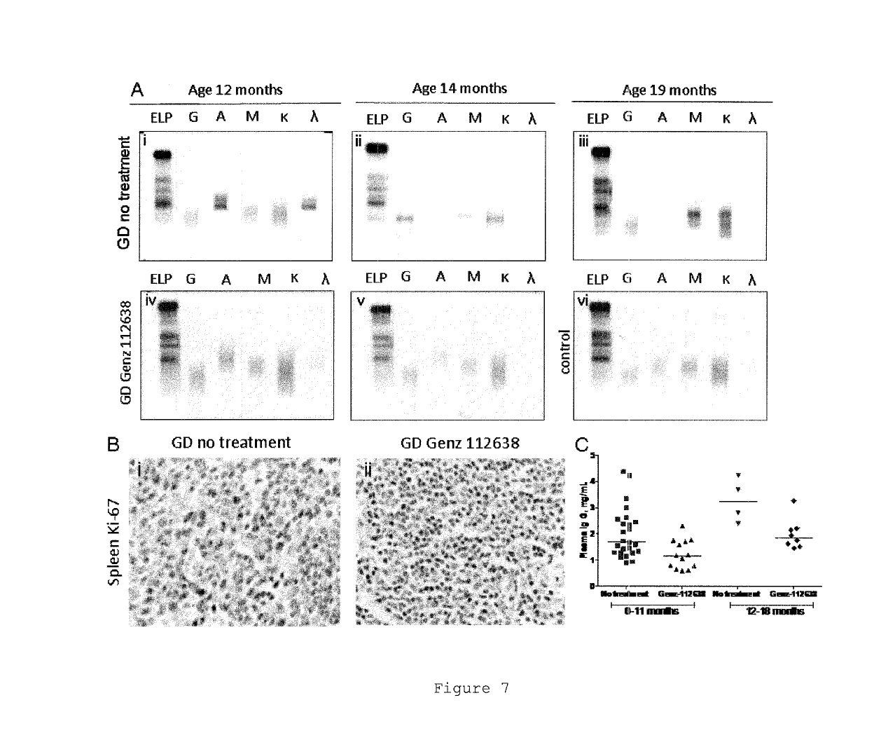

Histological sections of spleens from Gaucher disease mice commonly showed expansion of the red pulp with plasmacytosis. As previously reported, the normal splenic architecture was distorted with lymphoid hyperplasia (FIG. 6 ii, v) and manifest infiltration by large, lipid-engorged Gaucher cells [22]. Compared with untreated GD mice, in those that had received GENZ 112638, histological examination of haematoxylin and eosin stained sections showed preservation of the splenic architecture and reduced or absent inflammatory changes in the liver, lungs and kidneys—immuno-histological staining confirmed the presence of well-defined zones of B and T cell compartments in the follicles (FIG. 6 iii, vi). Immunostaining for the proliferation marker Ki-67 was increased in tissues with lymphoid hyperplasia in untreated GD mice. After treatment with Genz-112638, Ki-67 staining was less evident indicating decreased proliferative status (FIG. 7 B ii). Plasma-cell infiltration in the red pulp of the spleens was also less evident in the GD animals that had received GENZ 112638 than in age-matched GD mice that had not received the study drug (FIG. 6 viii, ix). We noted a slight decrease in the cellularity of the spleens obtained from Gaucher mice that had received GENZ 112638. The spleen and liver weights did not differ in treated and untreated Gaucher mice.

### Protein Electrophoresis and Immunofixation

Plasma/serum protein electrophoresis showed that none of the GD mice which had received Genz-112638 for at least 10 months from six weeks of age had any detectable paraprotein species (Table 1, FIG. 7A:4,5). In contrast, in 18 out of 48 age-matched GD mice that had not received treatment with GENZ 112638, monoclonal paraprotein species were readily visible after electrophoresis; their identity was confirmed by immunofixation (FIG. 7A:1, 2, 3). Total plasma immunoglobulin G concentrations were decreased significantly in animals that had received the study drug but in several untreated Gaucher disease mice, plasma IgG concentrations were increased beyond the appropriate reference control values (FIG. 7C).

Delayed Treatment with Genz-112638 and Frequency of Lymphoma in Gaucher Mice

We investigated preventive and therapeutic protocols: whether delayed administration of the active agent Genz-112638 would reverse the manifestations of disease that became established with age in GD mice. GD mice of mean age 7.3 months received GENZ 112638 at 300 mg/kg daily for a further 5.4 months. While the agent decreased the plasma concentrations of glucosylceramide and glucosylsphingosine, histological analysis showed established lymphoid hyperplasia in five mice and one with a frank splenic B cell lymphoma. Moreover, plasma protein electrophoresis and immunofixation showed the presence of a monoclonal paraprotein or oligoclonal immunoglobulin secretion in five out of the total of nine mice with Gaucher disease in this delayed treatment group (table 2).

Here, we report striking therapeutic effects of GENZ 112638 on the frequency of B cell lymphoma and monoclonal gammopathy as a preventive stratagem in a conditional murine Gaucher disease model. Early administration of GENZ 112638, a selective UDP-glucose: N-acylsphingosine transferase inhibitor, at 300 mg/kg completely prevented the occurrence of myeloma and B-cell lymphoma in the murine non-neuronopathic Gaucher disease model; the agent moreover suppressed the appearance of the signature biomarker of these B-cell cancers—development of monoclonal paraproteinaemia. The predicted effect of the study drug on the biochemical changes associated with the appearance of Gaucher disease was clearly observed, with a notable decrease in the elevated plasma concentrations of β-D-glucosylsphingosine, that characterize this conditional murine model of Gaucher disease. GENZ 112638 also prevented the florid appearance of pathological macrophages and the proliferation of lymphocytes in the spleen and other organs that characterizes this informative model [22, 41].

Late administration of GENZ 112638 after 7 months of age in mice with established accumulation of glycosphingolipids in organs and plasma did not completely prevent lymphoproliferation and development of B cell lymphoma. The inhibitory action of the agent on de novo synthesis of glycosphingolipids in established disease may require prolonged exposure of the drug to maintain a physiological balance.

Glucosylsphingosine is not detectable in blood or the organs of healthy individuals but it is greatly increased in plasma of individuals with Gaucher disease (19). It is notable in our prior study that GD animals with B-cell lymphoma were those with the highest mean plasma concentration of glucosylsphingosine; this metabolite was only modestly increased in the tissues [22]. Preferential inhibition of the formation of glucosylsphingosine by GENZ 112638 in this Gaucher disease murine model is consistent with a potential stimulatory action of this molecule on downstream signaling pathways that lead to systemic malignant proliferation of B cell precursors and lymphocytes.

These findings suggest that lipids other than the N-acyl-sphingosyl-1-O-β-D glucosides with varying acyl and sphingosine moieties (collectively termed β-D-glucosylceramides), which constitute the principal lysosomal storage material in the pathological macrophages of Gaucher disease, have unique signaling properties that directly or indirectly influence the behaviour of B lymphocytes and cognate plasma cells and drive malignant transformation.

GENZ 112638 serves as a ceramide analogue competitively to inhibit UDP-glucose: N-acylsphingosine transferase, an enzyme of the Golgi complex that catalyses formation of glucosylceramide from ceramide and UDP-glucose, in the first committed step in the formation of glucosylceramide-based sphingolipids. The molecule has been shown to have a highly specific inhibitory action towards this molecular target in vitro with minimal inhibitory properties towards glycosidases and other enzymes involved in sphingolipid metabolism [49].

The effect of inhibition of de novo synthesis of glucosylceramide on lymphocyte populations merits further consideration. The iminosugar, N-butyldeoxynojirimycin, a non-selective inhibitor of UDP-glucosylceramide synthase, is approved for individuals with Gaucher disease. Otherwise, healthy wild type C57BL/6 mice given N-butyldeoxynojirimycin at rising doses (600 to 2400 mg/kg) for a period of four months reduced the size of lymphoid organs without gross morphological changes. A small decrease in the ratio of B to T cell populations in spleens and thymuses was observed. [50]. An anti-proliferative action of inhibitors of UDP-glucosylceramine transferase (D-threo-1phenyl-2-decanoylamino-3morpholino-1-propanol analogues) has been demonstrated in several cancers [51, 52, 53, 54]. Moreover the inhibitor reduced clonogenic potential and viability of murine myeloma cells. GENZ 112638 has moreover been shown to a repress AKT protein kinase-mammalian target of rapamycin signaling and cell-cycle control that is dysregulated in polycystic kidney disease (40).

Here we describe a striking therapeutic effect of a potent and selective inhibitor of UDP-glucosylceramide transferase (GENZ 112638), on B cell lymphoma and myeloma in an experimental Gaucher disease model. This shows that disturbed sphingolipid homeostasis drives clonal expansion of B lymphocytes and their precursors [22]; our findings have clear potential for salutary suppressive effects on malignant transformation and strongly implicate sphingolipid metabolites in the regulation of B-cell biology, as well as supporting the use of systemic inhibitors of glycosphingolipid biosynthesis in Gaucher disease.

TABLE 1
Frequency of Monoclonal immunoglobulin species in
Gaucher mice

| | |
|---|---|
| GD No treatment | GD + Eliglustat† |

| Age group | M-protein* | Total | M-protein* | Total |
|---|---|---|---|---|
| 0-6 months | 2 | 4 | 0 | 4 |
| 7-12 months | 4 | 17 | 0 | 10 |
| 13-18 months | 8 | 20 | 0 | 8 |
| 19-24 months | 4 | 7 | — | — |
| Total | 18 | 48 | 0 | 22 |

†Eliglustat 300 mg/Kg daily months 1.5-12 inclusive

TABLE 2
Late treatment: Frequency of M protein species in GD mice

| | |
|---|---|
| No treatment | Eliglustat delayed† |

| Age group | M-protein* | Total | M protein* | Total |
|---|---|---|---|---|
| 12-18 months | 8 | 18 | 5 | 8 |

†Eliglustat 300 mg/Kg daily months 7-12 inclusive

TABLE 3
Lymphoma incidence in Gaucher mice

| | |
|---|---|
| GD No treatment | GD + Eliglustat† |

| Age group | Lymphoma* | Total | Lymphoma* | Total |
|---|---|---|---|---|
| 0-6 months | 2 | 8 | 0 | 4 |
| 7-12 months | 2 | 26 | 0 | 10 |
| 13-18 months | 7 | 20 | 0 | 8 |
| 19-24 months | 3 | 7 | — | — |
| Total | 14 | 61 | 0 | 22 |

TABLE 4

| | Lymphoid | Lymphoma/ | |
|---|---|---|---|
| | hyperplasia | myeloma | Total |
| No | 5 | 1 | 8 |
| treatment | | | |
| Eliglustat | 4 | 1 | 9 |
| delayed† | | | |

†Eliglustat 300 mg/Kg daily, Mean age 7.3 months Average length of treatment 5.4 months

### REFERENCES

1. National Cancer Institute. SEER cancer statistics review, 1975-2003.
2. de Fost M et al Blood Cells Mol Dis; 2006 36(1): 53-58.
3. Taddei T H et al Am J Hematol. 2009; 84(4):208-214
4. Rosenbloom B E et al. Blood 2005; 105(12):4569-4572
5. Benjamin D et al Scand J Haematol 1979; 22:179-184
6. Fox H et al Cancer 1984; 54:312-314
7. Garfinkel D et al Arch Intern Med 1982; 142:2229-2230
8. Harder H et al Ann Hematol 2000; 79:640-643
9. Weinreb N J. Crit Rev Oncog. 2013; 18(3):177-95
10. Allen M J et al QJM. 1997 January; 90(1):19-25
11. de Fost M et al Ann Hematol 2008; 87(6): 439-449.
12. Arends M et al Br J Haematol. 2013 June; 161(6):832-42
13. Cox T M. J Inherit Metab Dis 2001; 24 Suppl 2:106-21; discussion 87-8
14. Boot R G et al Blood 2004; 103(1):33-39
15. Moran M T et al Blood. 2000 Sep. 1; 96(5):1969-78
16. Boven L A et al Am J Clin Pathol 2004; 122(3):359-369.
17. Barak V et al. Eur Cytokine Network 1999; 10(2):205-210
18. Lichtenstein M et al Blood Cells Mol Dis 1997; 23(3):395-401
19. Dekker N et al Blood, 2011: 118 (16):e118-127
20. Lahiri S et al Cell Mol Life Sci. 2007 September; 64(17):2270-84
21. Radin N S. Glycoconj J. 1996 April; 13(2):153-7
22. Pavlova E V et al J Pathol. 2013 September; 231(1):88-97.
23. Radin N S et al Res Commun Chem Pathol Pharmacol. 1972 May; 3(3):637-44
24. Vunnam R R et al Chem Phys Lipids. 1980 April; 26(3):265-78
25. Cox T et al (2000) Lancet 355 9214 1481-1485
26. Elstein D et al. (2004) J Inherit Metab Dis.; 27(6):757-766
27. Cox T M et al 2003 J Inherit Metab Dis.; 26: 513-526
28. Cox T M, et al (2012) Orphanet Journal of Rare Diseases December 27; 7:102. doi: 10.1186/1750-1172-7-102.
29. Lee L et al (1999) J Biol Chem; 274:14662-14669
30. Shayman J A. (2010) Drugs Future; 35:613-620
31. McEachern K A et al Mol Genet Metab. 2007 July; 91(3):259-67
32. Marshall J et al (2010) J. Inherited Metabolic Disease 33: 281-289
33. Peterschmitt M J et al (2011) J Clin Pharmacol; 51: 695-705
34. Lukina E et al Blood; 116(20):4095-8
35. Erratum in: Blood. 2011 May 19; 117(20):5551].
36. Hannun Y A, Obeid L M. (2008) Nat Rev Mol Cell Biol; 9: 139-150
37. Abe A et al (1995) J Lipid Res; 36: 611-21
38. Chatterjee S et al. (2013 PLoS One May 9; 8 (5):e63726
39. Manning L S, Radin N S. (1999) Br J Cancer; 81: 952-958
40. Natoli T A et al Nat Med; 16:788-92.
41. Enquist I B et al Proc Natl Acad Sci USA 2006; 103(37): 13819-13824
42. Elmore S A. Toxicol Pathol. 2012; 40(2):148-56
43. Yamaguchi Y et al J Biochem. 1994 September; 116(3):704-10
44. Kanazawa T et al J Cell Biol, 2000; 149(4):943-50.
45. Im D S et al J Cell Biol, 2001; 153(2):429-34
46. Mizukami H et al J Clin Invest 2002; 109(9): 1215-1221.
47. Liu J, et al Proc Natl Acad Sci USA 2012; 109(25):10018-10023
48. Mistry P K et al Proc Natl Acad Sci USA 2010; 107(45):19473-19478
49. Cox T M. Curr Opin Investig Drugs. 2010 October; 11(10):1169-81
50. Platt F M, et al J Biol Chem. 1997 Aug. 1; 272(31):19365-72
51. Inokuchi J et al Cancer Lett. 1987 December; 38 (1-2):23-30
52. Radin N S, et al Biochem Pharmacol. 1988 Aug. 1; 37(15):2879-86
53. Radin N S, et al Adv Lipid Res. 1993; 26:183-213
54. Abe A, R et al J Lipid Res. 1995 March; 36(3):611-21
55. Cox T M. Philos Trans R Soc Lond B Biol Sci. 2003; 358(1433):967-973
56. Wu Y P, et al Hum Mol Genet. 2008; 17(15):2257-2264.
57. Spiegel S et al (2003) Nat Rev Mol Cell Biol 4: 397-407
58. Spiegel S et al (2000) Biochim Biophys Acta 1484: 107-116
59. Green J A, et al Nat Immunol, 2011; 12(7): 672-80.
60. Wang F et al (1999) J Biol Chem 274: 35343-35350
61. Schwab S R, et al Nat Immunol. 2007 December; 8(12):1295-301
62. Felding-Habermann B et al Biochemistry. 1990 Jul. 3; 29(26):6314-22
63. Lee L et al (1999) J Biol Chem 274, 14662-14669

1. A method of treating lymphoproliferative cancer comprising; administering a UDP-glucosylceramide synthase inhibitor to an individual in need thereof.

2-3. (canceled)

4. The method according to claim 1 wherein the cancer is B-cell lymphoma.

5. The method according to claim 1 wherein the cancer is myeloma.

6. The method according to claim 1 wherein the cancer is characterised by monoclonal gammopathy or paraproteinemia.

7. The method according to claim 1 wherein the individual is a human.

8. The method according to claim 1 wherein the UDP-glucosylceramide synthase inhibitor does not increase intracellular ceramide concentrations in the individual.

9. The method according to claim 1 wherein the UDP-glucosylceramide synthase inhibitor does not inhibit 1-O-acylceramide synthase.

10. The method according to claim 1 wherein the UDP-glucosylceramide synthase inhibitor is eliglustat tartrate.

11. The method according to claim 1 wherein the individual has Gaucher disease.

12. The method according to claim 1 wherein the individual does not have Gaucher disease.

13. The method according to claim 1 wherein the treatment is prophylactic or preventative.

14. The method according to claim 1 wherein the individual has been previously identified as having a lymphoproliferative cancer or previously identified as being at risk of having a lymphoproliferative cancer.

15. The method according to claim 1 wherein the method or treatment further comprises identifying the individual as having a lymphoproliferative cancer or being at risk of having a lymphoproliferative cancer.
